(19) United States
(12) Patent Application Publication
(10) Pub. No.: US 2022/0151481 A1
(43) Pub. Date: May 19, 2022

(54) VAGINAL SPECULUM

(71) Applicant: IWASAKI INDUSTRY INC., Yamato-koriyama-shi, Nara (JP)

(72) Inventor: Yoshihisa IWASAKI, Yamato-koriyama-shi (JP)

(73) Assignee: IWASAKI INDUSTRY INC., Yamato-koriyama-shi, Nara (JP)

(21) Appl. No.: 17/436,229

(22) PCT Filed: Mar. 6, 2020
(86) PCT No.: PCT/JP2020/009842

§ 371 Date: Sep. 3, 2021

(30) Foreign Application Priority Data
Mar. 6, 2019 (JP) 2019-040973

(51) Int. Cl. A61B 1/303; A61B 1/32

(52) U.S. Cl. CPC A61B 1/303; A61B 1/32

(57) ABSTRACT

A vaginal speculum capable of mitigating discomfort is provided. The vaginal speculum includes a first portion, a second portion, a first elastomer portion, and a second elastomer portion. The first portion includes a frame body portion, a first blade portion that is to be inserted into a vaginal cavity, and a first handle portion. The second portion includes a second handle portion, and a second blade portion that is to be inserted into the vaginal cavity. The first elastomer portion is made of an elastomer and is on an outer face of the first blade portion. The second elastomer portion is made of an elastomer and is on an outer face of the second blade portion. The second handle portion is pivotable relative to the first handle portion. The second blade portion is pivoted relative to the first blade portion in response to pivot of the second handle portion.

### TECHNICAL FIELD

The present invention relates to a vaginal speculum.

### BACKGROUND ART

A vaginal speculum is used to visually inspect or medically treat the inside of the vagina or uterus. A vaginal speculum is inserted into the vaginal cavity and widens the vaginal cavity. For example, Patent Literature 1 discloses a vaginal speculum with a configuration in which a pair of blades that can be separated from each other are connected such that they can be rotated relative to each other. One blade of this vaginal speculum is provided with ratchet teeth arranged in two rows, and the other blade is provided with a pawl that can selectively engage with the ratchet teeth. This vaginal speculum is provided with a means that connects the two rows of ratchet teeth and can move the pawl from one row to the other row.

### CITATION LIST

### Patent Literature

Patent Literature 1: JP S51-51188A

### SUMMARY OF INVENTION

### Technical Problem

The vaginal speculum disclosed in Patent Literature 1 is made of plastic (hard resin). Such a vaginal speculum is likely to cause discomfort such as pain to a subject when it is inserted into the vaginal cavity. Moreover, a conventionally used metallic vaginal speculum may cause sensation of cold to the vagina and thus cause discomfort to a subject.

It is an object of the present invention to provide a vaginal speculum capable of mitigating discomfort felt by a subject.

### Solution to Problem

A vaginal speculum according to an aspect of the present invention includes a first portion, a second portion, a first elastomer portion, and a second elastomer portion. The first portion includes an annular frame body portion that extends in a front-rear direction, a first blade portion that extends forward from the frame body portion and is to be inserted into a vaginal cavity, and a first handle portion that extends from the frame body portion in a radial direction. The second portion includes a second handle portion that is opposed to the first handle portion, and a second blade portion that extends forward from the second handle portion, is opposed to the first blade portion, and is to be inserted into the vaginal cavity together with the first blade portion. The first elastomer portion is made of an elastomer and is arranged on a first outer face of the first blade portion, the first outer face being on a side opposite to the second blade portion. The second elastomer portion is made of an elastomer and is arranged on a second outer face of the second blade portion, the second outer face being on a side opposite to the first blade portion. The second handle portion is configured to be pivotable relative to the first handle portion. The second blade portion is configured to be pivoted relative to the first blade portion while being relatively moved away therefrom and brought close thereto, in response to pivot of the second handle portion relative to the first handle portion. The first elastomer portion and the second elastomer portion are arranged to prevent the first outer face of the first blade portion and the second outer face of the second blade portion from coming into contact with an inner wall of the vaginal cavity when the first blade portion and the second blade portion are inserted into the vaginal cavity.

It should be noted that the “elastomer” is a general term for elastic macromolecular materials and encompasses rubbers and thermoelastic elastomers.

With the configuration mentioned above, the first elastomer portion made of an elastomer is arranged on the first outer face of the first blade portion to be inserted into the vaginal cavity. Also, the second elastomer portion made of an elastomer is arranged on the second outer face of the second blade portion to be inserted into the vaginal cavity together with the first blade portion. Accordingly, the first elastomer portion and the second elastomer portion prevent the first outer face of the first blade portion and the second outer face of the second blade portion from coming into contact with the inner wall of the vaginal cavity when the first blade portion and the second blade portion are inserted into the vaginal cavity. That is, pain, sensation of cold, and a feeling that a foreign material is present caused by the contact of the first outer face of the first blade portion and the second outer face of the second blade portion with the inner wall of the vaginal cavity are mitigated, and discomfort felt by a subject is thus mitigated. It should be noted that the first elastomer portion and the second elastomer portion need not completely prevent the vaginal speculum from coming into contact with the inner wall as long as they are arranged such that discomfort caused by the contact of the first outer face and the second outer face with the inner wall of the vaginal cavity can be mitigated.

In the vaginal speculum mentioned above, the first elastomer portion may include a protruding portion that protrudes forward with respect to a front end portion of the first blade portion.

In the vaginal speculum mentioned above, the second elastomer portion may include a protruding portion that protrudes forward with respect to a front end portion of the second blade portion.

In the vaginal speculum mentioned above, the first portion and the second portion may be made of a hard resin or a metal.

In the vaginal speculum mentioned above, the first elastomer portion may be formed to be inseparable from the first portion, and the second elastomer portion may be formed to be inseparable from the second portion.

In the vaginal speculum mentioned above, one of the first handle portion and the second handle portion may include a projection-bearing member that includes a main body portion extending in a front-rear direction and a projection formed on the main body portion. Moreover, the other of the first handle portion and the second handle portion may be provided with a through hole through which the projection-bearing member passes, and a locking member that is to be engaged with the projection to lock a pivoted position of the second blade portion relative to the first blade portion.

With the configuration mentioned above, it is possible to lock the pivoted position of the second blade portion relative to the first blade portion, and therefore, the workability of using the vaginal speculum is improved.

In the vaginal speculum mentioned above, at least one of the projection-bearing member and the locking member may be made of a soft resin.

With the configuration mentioned above, it is easy to elastically deform at least one of the projection-bearing member and the locking member, and thus the operability is improved and impact on the hand is reduced.

In the vaginal speculum mentioned above, the first blade portion may include a first protrusion constituted by a bulging portion formed on the first outer face, the second blade portion may include a second protrusion constituted by a bulging portion formed on the second outer face, the first elastomer portion may be arranged on a region on the first outer face other than the first protrusion and be brought into intimate contact with a side peripheral portion of the first protrusion, and the second elastomer portion may be arranged on a region on the second outer face other than the second protrusion and be brought into intimate contact with a side peripheral portion of the second protrusion.

### Advantageous Effects of the Invention

With the aspects described above, the first elastomer portion and the second elastomer portion prevent the first outer face of the first blade portion and the second outer face of the second blade portion from coming into contact with the inner wall of the vaginal cavity when the first blade portion and the second blade portion are inserted into the vaginal cavity. Accordingly, pain, sensation of cold, and a feeling that a foreign material is present caused by the contact of the first blade portion and the second blade portion with the inner wall of the vaginal cavity are mitigated, and discomfort felt by a subject is thus mitigated.

### BRIEF DESCRIPTION OF DRAWINGS

FIG. 1A is a perspective view of a vaginal speculum as viewed from a rear portion side.

FIG. 1B is a perspective view of the vaginal speculum as viewed from a front portion side.

FIG. 2 is a perspective view of the rear portion (first portion).

FIG. 3 is a perspective view of the front portion (second portion).

FIG. 4A is a perspective view of a locking member.

FIG. 4B is a perspective view of the locking member.

FIG. 5 is a perspective view of a projection-bearing member.

FIG. 6 is a perspective view showing the open state of the vaginal speculum.

FIG. 7 is a perspective view of a vaginal speculum according to another embodiment.

FIG. 8 is an exploded perspective view of the vaginal speculum according to another embodiment.

FIG. 9 is another perspective view of the vaginal speculum according to another embodiment.

FIG. 10 is another exploded perspective view of the vaginal speculum according to another embodiment.

FIG. 11 is a perspective view showing the locked state of the vaginal speculum.

FIG. 12 is a side view showing the open state of the vaginal speculum.

FIG. 13 is a back view showing the locked state of the vaginal speculum.

### DESCRIPTION OF EMBODIMENTS

Hereinafter, a vaginal speculum according to an embodiment of the present invention will be described with reference to the drawings.

### 1-1. Configuration of Vaginal Speculum According to First Embodiment

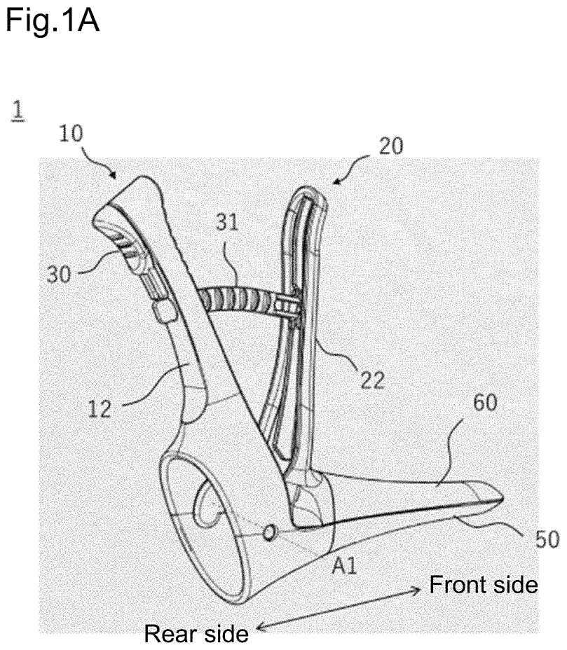

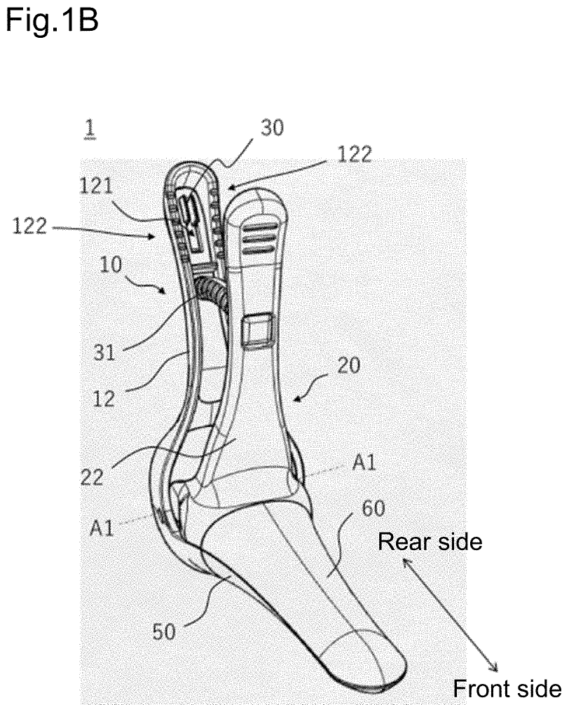

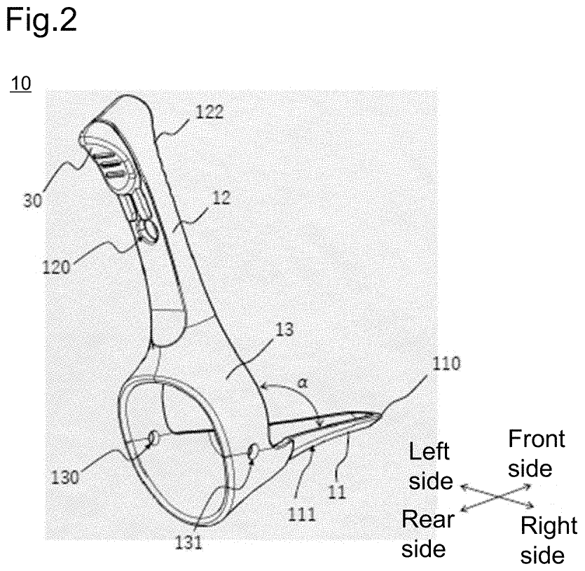

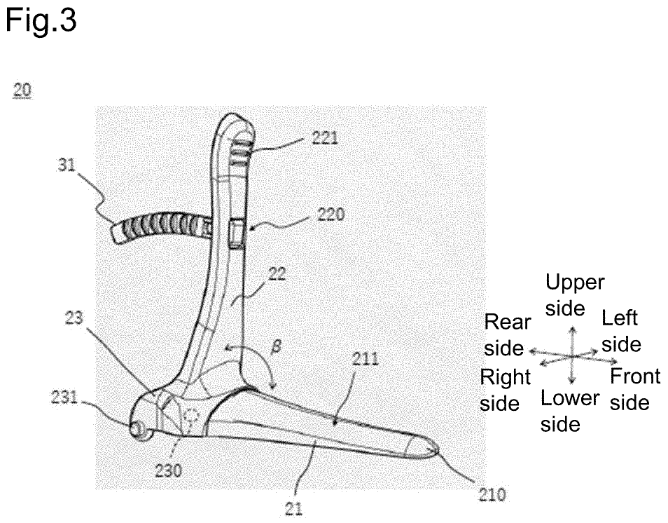

FIGS. 1A and 1B are perspective views of a vaginal speculum 1 according to a first embodiment. As shown in FIG. 1, the vaginal speculum 1 includes a rear portion (first portion) 10 that is arranged on the rear side, and a front portion (second portion) 20 that is arranged on the front side. The rear portion 10 includes a first blade portion 11 (see FIG. 2). The front portion 20 includes a second blade portion 21 (see FIG. 3). The first blade portion 11 and the second blade portion 21 are to be inserted into the vaginal cavity. The vaginal speculum 1 further includes a first elastomer portion 50 that is made of an elastomer and is arranged on a first outer face 111 of the first blade portion 11, and a second elastomer portion 60 that is made of an elastomer and is arranged on a second outer face 211 of the second blade portion 21.

FIG. 2 is a perspective view of the rear portion 10. As shown in FIG. 2, the rear portion 10 includes a frame body portion 13 having an annular shape or a substantially cylindrical shape on the rear side. The frame body portion 13 extends in the front-rear direction shown in FIG. 2, and a first handle portion 12 and the first blade portion 11 are continuous with the front side of the frame body portion 13. The frame body portion 13 is provided with a through hole 130 and a through hole 131 on both sides in the left-right direction shown in FIG. 2. The through hole 130 and the through hole 131 are formed at positions that are opposed to each other, and receive a protrusion 230 and a protrusion 231 of the front portion 20, which will be described later, respectively. The rear portion 10 and the front portion 20 are thus coupled to each other. It is preferable that the rear portion 10 is made of a hard resin, and it is more preferable that the rear portion 10 is transparent.

The rear portion 10 further includes the first blade portion 11. The first blade portion 11 is to be inserted into the vaginal cavity, and is continuous with the inner and outer faces of the frame body portion 13 and extends forward therefrom. The first blade portion 11 has such a shape that is recessed toward the center from the end portions in the left-right direction shown in FIG. 2. Moreover, the first blade portion 11 is formed in a tapered shape such that the width in the left-right direction decreases from a rear end portion toward a front end portion 110. The front end portion 110 is formed in a rounded shape.

The rear portion 10 further includes the first handle portion 12. The first handle portion 12 is held by a hand of a user of the vaginal speculum 1 when the vaginal speculum 1 is handled. The first handle portion 12 is continuous with the front end of the frame body portion 13 and extends therefrom in the radial direction. On the side peripheral portion of the frame body portion 13, the position at which the first handle portion 12 is continuous therewith is opposed to the position at which the first blade portion 11 is continuous therewith and extends therefrom. In this embodiment, a configuration is employed in which an angle α between the first blade portion 11 and the first handle portion 12 about a coupling portion of the frame body portion 13 is larger than an angle β between a second blade portion 21 and a second handle portion 22, which will be described later.

The first handle portion 12 is provided with a through hole 120 near the center in the longitudinal direction. A projection-bearing member 31, which will be described later, is configured to pass through the through hole 120. The first handle portion 12 further includes a locking member 30. The locking member 30 and a projection-bearing member 31, which will be described later, form a locking mechanism for locking the pivoted position of the second blade portion 21 relative to the first blade portion 11. The configuration and operation of the locking mechanism will be described later. Furthermore, a plurality of grooves are formed at the edge on the front side of an end portion of the first handle portion 12, on a side opposite to the frame body portion 13. The plurality of grooves form an anti-slip portion 122 that exhibits an anti-slip effect when a user holds the first handle portion 12. There is no limitation to the configuration in which the anti-slip portion 122 is formed by grooves, and the anti-slip portion 122 may also be formed by protrusions or a combination of recesses and protrusions. Moreover, the anti-slip portion 122 may also be formed by fixing a member made of an anti-slip material such as an elastomer.

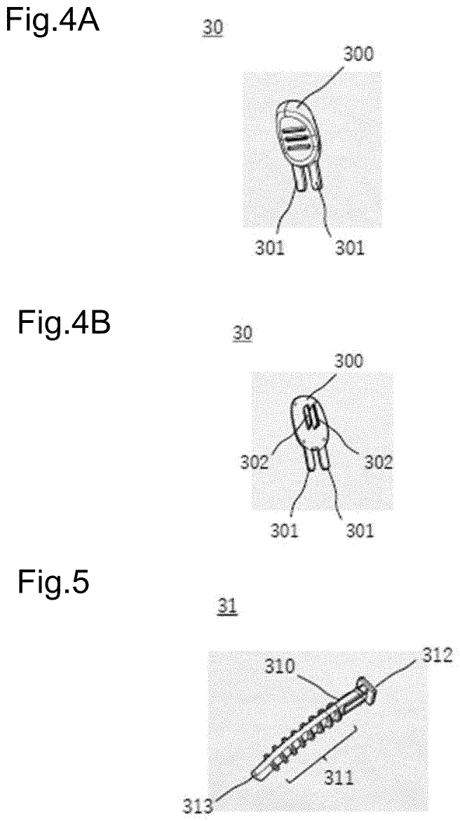

The locking member 30 is arranged on the rear side of the end portion of the first handle portion 12, on a side opposite to the frame body portion 13. FIG. 4A is a perspective view of the locking member 30 as viewed from the rear side, and FIG. 4B is a perspective view of the locking member 30 as viewed from the front side. The locking member 30 includes a main body portion 300, and two rod-like portions 301 that extend from one end of the main body portion 300. The main body portion 300 has a substantially elliptical shape.

As shown in FIG. 4A, the rear face of the main body portion 300 is gradually recessed toward the central portion and is provided with recesses and protrusions thereon for catching a finger. Moreover, the two rod-like portions 301 are arranged at a predetermined interval. As shown in FIG. 4B, the front face of the main body portion 300 is provided with two elongated protrusions 302. The elongated protrusions 302 are engaged in a rectangular through hole 121 formed in the first handle portion 12 (see FIG. 1B). The locking member 30 can be moved in the rectangular through hole 121 in the longitudinal direction while being engaged in the rectangular through hole 121. More specifically, the locking member 30 can be slid so as to be switched from a state in which the two rod-like portions 301 are farther away from the frame body portion 13 than the through hole 120 is, to a state in which the gap between the rod-like portions 301 overlaps the through hole 120. The locking member 30 is preferably made of a soft resin.

FIG. 3 is a perspective view of the front portion 20. The front portion 20 is arranged on the front side of the rear portion 10 and is coupled to the rear portion 10. The front portion 20 includes a second blade portion 21 and a second handle portion 22. The second handle portion 22 is opposed to the first handle portion 12 as shown in FIG. 1, and extends in the vertical direction shown in FIG. 3. The lower end portion of the second handle portion 22 is provided with a coupling portion 23, and the second blade portion 21 extends forward from the coupling portion 23. In this embodiment, the front portion 20 is configured such that the second blade portion 21 and the second handle portion 22 form an angle β about the coupling portion 23. As described above, the angle β is smaller than α. It is preferable that the front portion 20 is made of a hard resin, and it is more preferable that the front portion 20 is transparent.

The coupling portion 23 includes columnar protrusions 230 and 231 at the rear end portion. The protrusions 230 and 231 protrude in the left-right direction shown in the FIG. 3 and are provided at positions opposed to each other. The protrusion 230 is pivotably received by the through hole 130 of the rear portion 10. The protrusion 231 is pivotably received by the through hole 131 of the rear portion 10. A configuration is thus possible in which the rear portion 10 and the front portion 20 are coupled to each other, and the second handle portion 22 can be pivoted relative to the first handle portion 12 about an axis A1 shown in FIG. 1. With this configuration, the second blade portion 21 is pivoted relative to the first blade portion 11 while being relatively moved away therefrom and brought close thereto, in response to pivot of the second handle portion 22 relative to the first handle portion 12.

The second blade portion 21 extend forward from the coupling portion 23 (second handle portion 22) so as to face the first blade portion 11. The second blade portion 21 is to be inserted into the vaginal cavity together with the first blade portion 11. The second blade portion 21 has such a shape that is recessed toward the center from the end portions in the left-right direction shown in FIG. 3. Moreover, the second blade portion 21 is formed in a tapered shape such that the width in the left-right direction decreases from a rear end portion toward a front end portion 210. The front end portion 210 of the second blade portion 21 is formed in a rounded shape.

The second handle portion 22 is held by a hand of a user of the vaginal speculum 1 together with the first handle portion 12 when the vaginal speculum 1 is handled. An uneven portion 221 for catching a finger is provided on the front face of the upper end portion of the second handle portion 22. The uneven portion 221 may be formed by at least one of a groove and an elongated protrusion. Moreover, the uneven portion 221 may also be formed by arranging a material of a member different from the second handle portion 22, for example. The second handle portion 22 is provided with a through hole 220 located near the center in the longitudinal direction, and includes a projection-bearing member 31 that is fixed inside the through hole 220 and extends rearward. The side peripheral portion of a front end portion 312 (see FIG. 5) of the projection-bearing member 31 is fixed to the inner peripheral portion of the through hole 220 so as not to be capable of being separated therefrom.

FIG. 5 is a perspective view of the projection-bearing member 31. The projection-bearing member 31 includes a main body portion 310, and a plurality of projections 311 provided on the main body portion 310. The main body portion 310 extends in the front-rear direction shown in FIG. 1A, and includes a front end portion 312 and a rear end portion 313. The projections 311 are provided between the front end portion 312 and the rear end portion 313 at predetermined intervals.

The projections 311 are each formed in a disk shape around the axis in the longitudinal direction of the main body portion 310, and are configured to be capable of passing through the through hole 120 of the first handle portion 12. Moreover, the projections 311 each are configured to have an outer diameter larger than the interval between the two rod-like portions 301 of the locking member 30 and not to pass through the gap between the two rod-like portions 301. The main body portion 310 has a diameter smaller than the interval between the two rod-like portions 301 of the locking member 30. The projection-bearing member 31 is preferably made of a soft resin.

As shown in FIG. 1, the vaginal speculum 1 further includes a first elastomer portion 50. The first elastomer portion 50 is made of an elastomer and is arranged on a first outer face 111 of the first blade portion 11. It should be noted that, as shown in FIG. 2, the first outer face 111 is a face of the first blade portion 11 on a side opposite to the second blade portion 21. The first elastomer portion 50 is formed to be inseparable from the first blade portion 11, and covers the first outer face 111 of the first blade portion 11. With this configuration, the first elastomer portion 50 prevents the first outer face 111 of the first blade portion 11 from coming into contact with the inner wall of the vaginal cavity when the first blade portion 11 is inserted into the vaginal cavity.

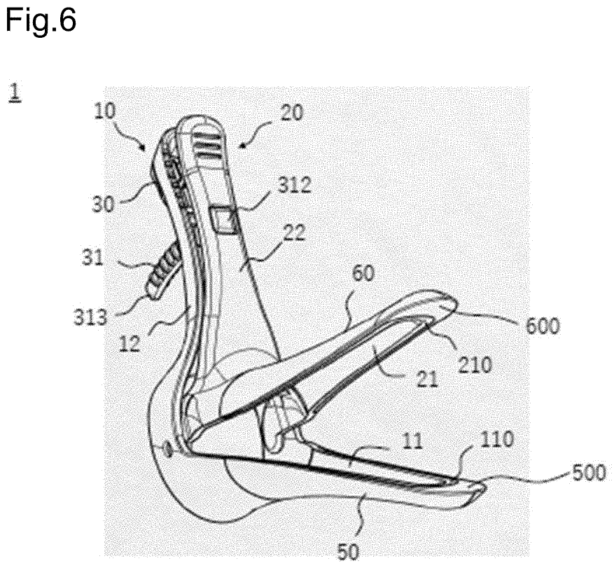

As shown in FIG. 6, the first elastomer portion 50 of this embodiment includes a protruding portion 500 that protrudes forward with respect to the front end portion 110 of the first blade portion 11. That is, a portion of the first elastomer portion 50 located at the front end portion 110 of the first blade portion 11 may be thicker than the other portions thereof. With this configuration, the first elastomer portion 50 prevents the front end portion 110 of the first blade portion 11 from coming into contact with the inner wall of the vaginal cavity when the first blade portion 11 is inserted into the vaginal cavity.

As shown in FIG. 1, the vaginal speculum 1 further includes a second elastomer portion 60. The second elastomer portion 60 is made of an elastomer and is arranged on a second outer face 211 of the second blade portion 21. It should be noted that, as shown in FIG. 3, the second outer face 211 is a face of the second blade portion 21 on a side opposite to the first blade portion 11. The second elastomer portion 60 is formed to be inseparable from the second blade portion 21, and covers the second outer face 211 of the second blade portion 21. With this configuration, the second elastomer portion 60 prevents the second outer face 211 of the second blade portion 21 from coming into contact with the inner wall of the vaginal cavity when the second blade portion 21 is inserted into the vaginal cavity.

As shown in FIG. 6, the second elastomer portion 60 of this embodiment includes a protruding portion 600 that protrudes forward with respect to the front end portion 210 of the second blade portion 21. That is, a portion of the second elastomer portion 60 located at the front end portion 210 of the second blade portion 21 may be thicker than the other portions thereof. With this configuration, the second elastomer portion 60 prevents the front end portion 210 of the second blade portion 21 from coming into contact with the inner wall of the vaginal cavity when the second blade portion 21 is inserted into the vaginal cavity.

When the rear portion 10 (it should be noted that the locking member 30 or the projection-bearing member 31 is excluded) is made of a hard resin, the rear portion 10 and the first elastomer portion 50 can be integrally molded through double molding, for example. First, a hard resin material is injected into a primary mold for molding the rear portion 10, and then the primary mold is opened. Next, the molded rear portion 10 is transferred to a secondary mold for molding the first elastomer portion 50. An elastomer raw material used for molding the first elastomer portion 50 is injected into the secondary mold, and then the elastomer raw material is cross-linked. Then, the secondary mold is opened, and the rear portion 10 and the first elastomer portion 50 are obtained as one piece.

Similarly, when the front portion 20 (it should be noted that the locking member 30 or the projection-bearing member 31 is excluded) is made of a hard resin, the front portion 20 and the second elastomer portion 60 can be integrally molded through double molding, for example. First, a hard resin material is injected into a primary mold for molding the front portion 20, and then the primary mold is opened. Next, the molded front portion 20 is transferred to a secondary mold for molding the second elastomer portion 60. An elastomer raw material used for molding the second elastomer portion 60 is injected into the secondary mold, and then the elastomer raw material is cross-linked. Then, the secondary mold is opened, and the front portion 20 and the second elastomer portion 60 are obtained as one piece.

### 1-2. Configuration of Vaginal Speculum According to Second Embodiment

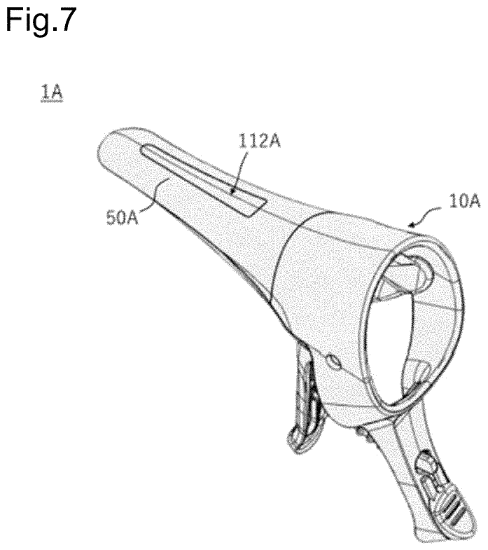

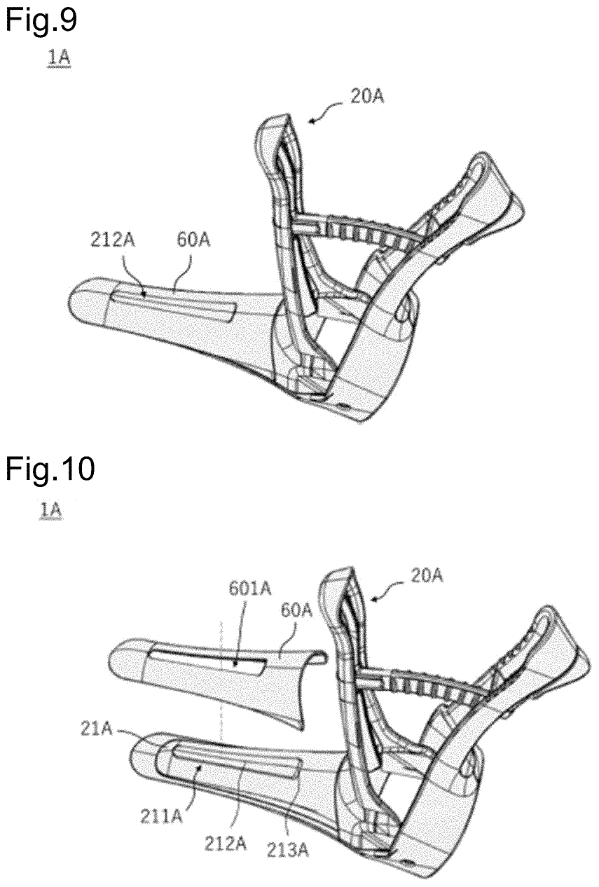

Hereinafter a vaginal speculum 1A according to a second embodiment will be described. As shown in the perspective views of FIGS. 7 and 9, the vaginal speculum 1A according to the second embodiment has the same configuration as that of the vaginal speculum 1 according to the first embodiment, except that a first outer face 111A and a second outer face 211A are partially exposed from a first elastomer portion 50A and a second elastomer portion 60A, respectively. Therefore, only configurations different from those of the vaginal speculum 1 will be described below, and descriptions of the same configurations as those of the vaginal speculum 1 will be omitted.

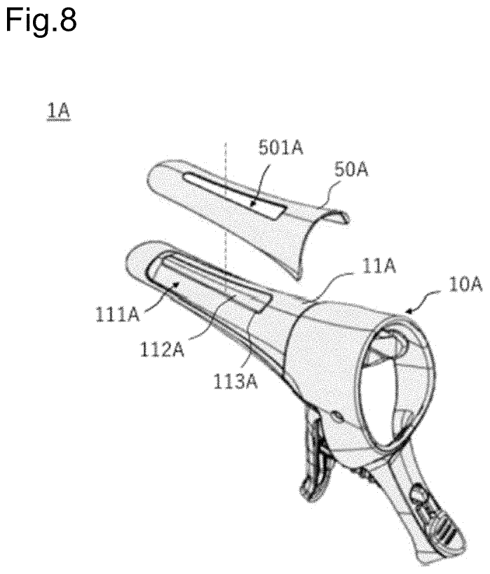

FIG. 7 is a perspective view of the vaginal speculum 1A as viewed from the first elastomer portion 50A side, and FIG. 8 is an exploded perspective view showing a state in which a first blade portion 11A and the first elastomer portion 50A are separated from each other. As shown in FIG. 7, the upper face with a substantially rectangular shape of a first protrusion 112A (a portion of the first outer face 111A) is seen in the surface of the first elastomer portion 50A. As shown in FIG. 8, the first protrusion 112A is constituted by a bulging portion formed on the first outer face 111A, and includes a first side peripheral portion 113A. The first elastomer portion 50A is arranged on a region on the first outer face 111A other than the first protrusion 112A and comes into intimate contact with the first side peripheral portion 113A. Accordingly, as shown in FIG. 8, the first elastomer portion 50A is provided with an opening 501A with substantially the same shape as the planar shape defined by the first side peripheral portion 113A. It should be noted that, as shown in FIG. 7, the first side peripheral portion 113A cannot be visually recognized from the outside of the vaginal speculum 1A. Moreover, the first elastomer portion 50A is arranged such that the surface thereof is flush with the upper face of the first protrusion 112A.

With this configuration, when a rear portion 10A it should be noted that the locking member 30 or the projection-bearing member 31 is excluded) and the first elastomer portion 50A are molded through double molding described above, a secondary mold can be formed such that the first protrusion 112A comes into contact with the inner wall of the secondary mold. If an elastomer raw material is injected into the secondary mold in the state in which the first protrusion 112A is in contact with the inner wall of the secondary mold, the elastomer raw material is more likely to be distributed inside the secondary mold. Accordingly, the first elastomer portion 50A can be uniformly formed on the region on the first outer face 111A other than the first protrusion 112A, and the unity of the first blade portion 11A and the first elastomer portion 50A is further improved.

FIG. 9 is a perspective view of the vaginal speculum 1A as viewed from the second elastomer portion 60A side, and FIG. 10 is an exploded perspective view showing a state in which a second blade portion 21A and the second elastomer portion 60A are separated from each other. As shown in FIG. 9, the upper face with a substantially rectangular shape of a second protrusion 212A (a portion of the second outer face 211A) is seen in the surface of the second elastomer portion 60A. As shown in FIG. 10, the second protrusion 212A is constituted by a bulging portion formed on the second outer face 211A, and includes a second side peripheral portion 213A. The second elastomer portion 60A is arranged on a region on the second outer face 211A other than the second protrusion 212A and comes into intimate contact with the second side peripheral portion 213A. Accordingly, as shown in FIG. 10, the second elastomer portion 60A is provided with an opening 601A with substantially the same shape as the planar shape defined by the second side peripheral portion 213A. It should be noted that, as shown in FIG. 9, the second side peripheral portion 213A cannot be visually recognized from the outside of the vaginal speculum 1A. Moreover, the second elastomer portion 60A is arranged such that the surface thereof is flush with the upper face of the second protrusion 212A.

With this configuration, when a front portion 20A (it should be noted that the locking member 30 or the projection-bearing member 31 is excluded) and the second elastomer portion 60A are molded through double molding described above, a secondary mold can be formed such that the second protrusion 212A comes into contact with the inner wall of the secondary mold. If an elastomer raw material is injected into the secondary mold in the state in which the second protrusion 212A is in contact with the inner wall of the secondary mold, the elastomer raw material is more likely to be distributed inside the secondary mold. Accordingly, the second elastomer portion 60A can be uniformly formed on the region on the second outer face 211A other than the second protrusion 212A, and the unity of the second blade portion 21A and the second elastomer portion 60A is further improved.

### 2. Operation of Vaginal Speculum

Hereinafter, the operation of the vaginal speculum according to the embodiment of the present invention will be described with reference to the drawings. It should be noted that the operation of the vaginal speculum 1A is the same as that of the vaginal speculum 1, and therefore, the vaginal speculum 1 is taken as an example and described below.

FIG. 1 shows the vaginal speculum 1 in a state in which the second handle portion 22 is not pivoted relative to the first handle portion 12. That is, the state shown in FIG. 1 is a closed state in which the first blade portion 11 and the second blade portion 21 face each other and fit together, there is no gap therebetween, and the vaginal speculum 1 is closed. In the closed state, the first handle portion 12 and the second handle portion 22 are separated at approximately an angle corresponding to a value obtained by subtracting β from α, about the axis A1. Moreover, none of the plurality of projections 311 of the projection-bearing member 31 pass through the through hole 120 of the first handle portion 12 from the front side to the rear side, and the projections 311 are located between the second handle portion 22 and the first handle portion 12.

A user inserts the first blade portion 11 and the first elastomer portion 50 as well as the second blade portion 21 and the second elastomer portion 60 into the vaginal cavity while keeping the vaginal speculum 1 in the closed state. The first blade portion 11, the first elastomer portion 50, the second blade portion 21, and the second elastomer portion 60 are inserted to an appropriate position inside the vaginal cavity.

Subsequently, the user pivots the second handle portion 22 relative to the first handle portion 12. The second blade portion 21 is moved away from the first blade portion 11 in response to the relative pivot of the second handle portion 22, and is pivoted relative to the first blade portion 11. As a result, a gap is formed between the first blade portion 11 and the second blade portion 21, so that the vaginal speculum 1 is brought into an open state as shown in FIG. 6, and the vaginal cavity is widened. When relatively pivoting the second handle portion 22, the user can pivot the second handle portion 22 by holding the locking member 30 of the first handle portion 12 and the uneven portion 221 of the second handle portion 22 to bring the second handle portion 22 close to the first handle portion 12.

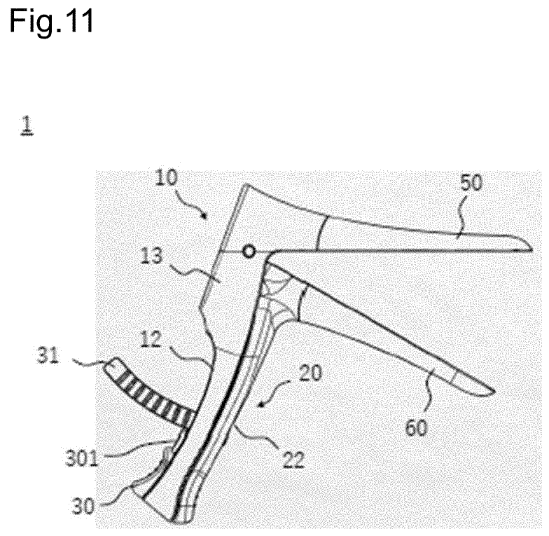

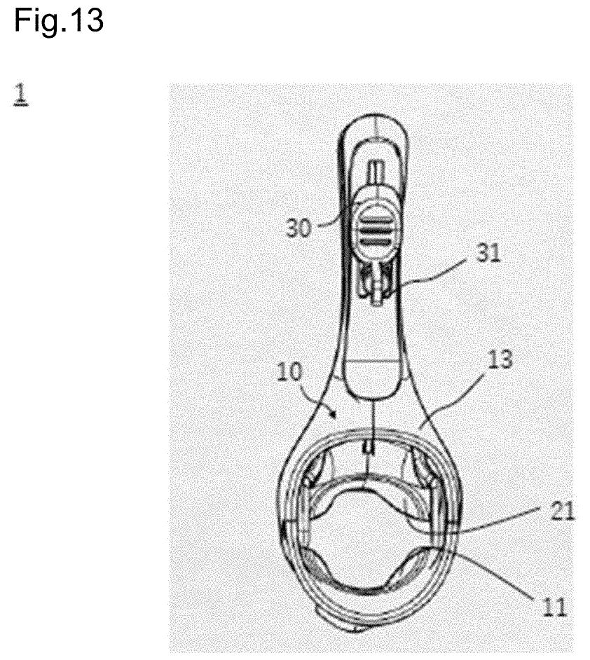

The projections 311 of the projection-bearing member 31 pass through the through hole 120 of the first handle portion 12 from the front side to the rear side as the second handle portion 22 is brought close to the first handle portion 12. The user can use the locking mechanism including the locking member 30 and the projection-bearing member 31 in order to fix the second handle portion 22 at an appropriate relative pivoted position. More specifically, in the state shown in FIG. 6, the user slides the locking member 30 toward the frame body portion 13, and thus the two rod-like portions 301 of the locking member 30 is inserted into a gap between the adjacent projections 311 of the projection-bearing member 31. That is, the locking member 30 is engaged with the projections 311. As a result, the position of the projection-bearing member 31 (second handle portion 22) with respect to the first handle portion 12 is fixed, and the vaginal speculum 1 is brought into a locked state shown in FIGS. 11 and 13.

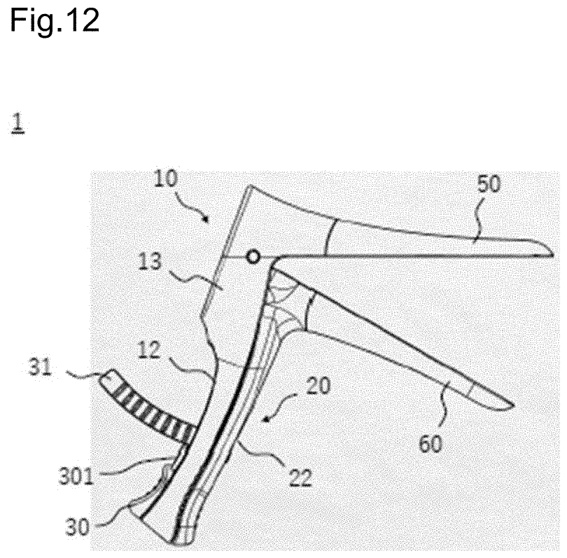

The projection-bearing member 31 includes the plurality of projections 311. Accordingly, the user of the vaginal speculum 1 can select the relative pivoted position of the second blade portion 21 to be locked in a stepwise manner. The vaginal speculum 1 of this embodiment is configured such that, when the second blade portion 21 is pivoted to the maximum, the first handle portion 12 and the second handle portion 22 abut against each other. At this time, an angle between the first handle portion 12 and the second handle portion 22 about the axis A1 is 0 degree (see FIG. 12).

In the open state of the vaginal speculum 1, the second blade portion 21 is pivoted relative to the first blade portion 11 and is moved away from the first blade portion 11. As a result, the user can observe the front side with respect to the vaginal speculum 1 through the frame body portion 13 (see FIG. 13).

In the locked state of the vaginal speculum 1, when the locking member 30 is slid in a direction away from the frame body portion 13, the locked state of the vaginal speculum 1 is cancelled. That is, the state in which the locking member 30 is engaged with the projections 311 of the projection-bearing member 31 is cancelled, and thus the second handle portion 22 can be pivoted relative to the first handle portion 12. When the user pivots the second handle portion 22 relative to the first handle portion 12 such that the second handle portion 22 is moved away from the first handle portion 12, the second blade portion 21 is pivoted in response to this pivot, and is relatively brought close to the first blade portion 11. The vaginal speculum 1 can be returned to the closed state shown in FIG. 1 by relatively pivoting the second handle portion 22 till the second blade portion 21 and the first blade portion 11 fit together.

### 3. Features

As described above, in the vaginal speculum 1, the first elastomer portion 50 and the second elastomer portion 60 are respectively arranged on the first outer face 111 of the first blade portion 11 and the second outer face 211 of the second blade portion 21, the first blade portion 11 and the second blade portion 21 being to be inserted into the vaginal cavity. The first elastomer portion 50 and the second elastomer portion 60 prevent the first outer face 111 of the first blade portion 11 and the second outer face 211 of the second blade portion 21 from coming into contact with the inner wall of the vaginal cavity when the first blade portion 11 and the second blade portion 21 are inserted into the vaginal cavity. Accordingly, the first elastomer portion 50 and the second elastomer portion 60, which are made of elastic elastomer, mitigate sensation of cold, pain, and a feeling that a foreign material is present caused by the contact of the first outer face 111 of the first blade portion 11 and the second outer face 211 of the second blade portion 21 with the inner wall of the vaginal cavity.

It should be noted that, also in the vaginal speculum 1A, the first elastomer portion 50A is arranged on the region on the first outer face 111A other than the first protrusion 112A, and the second elastomer portion 60A is arranged on the region on the second outer face 211A other than the second protrusion 212A. Accordingly, the entire first outer face 111A and the entire second outer face 211A are prevented from coming into contact with the inner wall of the vaginal cavity, and thus sensation of cold, pain, and a feeling that a foreign material is present caused by the contact of the entire first outer face 111A and the entire second outer face 211A with the inner wall of the vaginal cavity is significantly mitigated.

With the vaginal speculum 1, the locking mechanism that includes the locking member 30 and the projection-bearing member 31 can be used to lock the pivoted position of the second blade portion 21 relative to the first blade portion 11. Accordingly, a user need not keep on maintaining the relative pivoted position of the second blade portion 21, and thus the workability of using the vaginal speculum 1 is improved. It should be noted that the same applies to the vaginal speculum 1A.

In some of conventional vaginal specula, a locking mechanism for keeping the vaginal speculum in an open state is of a screw type that includes a bolt and a nut. The screw-type locking mechanism has a problem in that the screw threads of the bolt and the nut are likely to be misaligned, and the bolt and the nut fail to mesh with each other, and thus cannot be moved. Moreover, with the screw-type locking mechanism, it is necessary to move the nut to an appropriate position by rotating it in order to perform locking and to move the nut the same distance again by rotating it in order to perform unlocking, and normal operation is complicated. However, with locking mechanism included in the vaginal speculum 1 and the vaginal speculum 1A above, irrespective of the pivoted position (pivoted angle), locking and unlocking can be performed by sliding the locking member 30 a certain distance in the radial direction. Accordingly, a user can easily operate the locking mechanism included in the vaginal speculum 1 and the vaginal speculum 1A.

The locking member 30 and the projection-bearing member 31 are made of a soft resin, are elastic, and are easy to elastically deform. Accordingly, it is easy to engage the locking member 30 with the projection-bearing member 31 and to cancel the engagement therebetween. Moreover, impact on the hand of a user can be reduced, and thus a comfortable feeling can be given.

Furthermore, when using the vaginal speculum 1, a user mainly holds a portion near the locking member 30 of the first handle portion 12. The locking member 30 is made of a soft resin and thus is less likely to slip on the hand of the user, which enables more appropriate operation. Furthermore, the rear face of the locking member 30 is provided with recesses and protrusions, and the second handle portion 22 is provided with the uneven portion 221. This improves ease of gripping the vaginal speculum 1. It should be noted that the same applies to the vaginal speculum 1A.

The rear face of the main body portion 300 of the locking member 30 is gradually recessed toward the central portion in a side view. Accordingly, the locking member 30 is likely to catch a finger of a user, and thus the user can smoothly hold the vaginal speculum 1 and the vaginal speculum 1A and operate the locking mechanism.

### 4. Modified Examples

Although embodiments of the present invention have been described above, the present invention is not limited to the embodiments above, and various modifications can be carried out without departing from the gist of the invention. For example, the following modifications can be carried out.

4-1

In the embodiments above, the first portion and the second portion are configured such that the positional relationship is obtained in which the first handle portion 12, the second handle portion 22, the second blade portion 21, and the first blade portion 11 are arranged in this order in the clockwise direction about the axis A1. However, there is no limitation to the configuration in which the rear portion 10 and the front portion 20 are used as the first portion and the second portion as long as the first handle portion 12 and the second handle portion 22 can be pivoted relative to each other, and in response to this, the first blade portion 11 and the second blade portion 21 can be pivoted relative to each other such that these blade portions are relatively moved away from and brought close to each other. For example, the first portion and the second portion may be configured such that the positional relationship is obtained in which the second handle portion 22, the first handle portion 12, the second blade portion 21, and the first blade portion 11 are arranged in this order in the clockwise direction about the axis A1.

4-2

In the embodiments above, the portions of the rear portion 10 and the front portion 20 other than the locking member 30 and the projection-bearing member 31 are made of a hard resin. However, the rear portion 10 and the front portion 20 may be made of a metal. Moreover, the rear portion 10 and the front portion 20 may be made of different materials.

4-3

In the embodiments above, the locking member 30 and the projection-bearing member 31 are made of a soft resin. However, the locking member 30 and the projection-bearing member 31 may be made of a hard resin or the like. Moreover, the locking member 30 and the projection-bearing member 31 may be made of different materials.

4-4

In the embodiments above, the first portion (rear portion 10) includes the locking member 30, and the second portion (front portion 20) includes the projection-bearing member 31. However, the first portion (rear portion 10) may include the projection-bearing member 31, and the second portion (front portion 20) may include the locking member 30.

4-5

The rear portion 10 and the first elastomer portion 50 of the first embodiment may be replaced by the rear portion 10A and the first elastomer portion 50A of the second embodiment. Moreover, the front portion 20 and the second elastomer portion 60 of the first embodiment may be replaced by the front portion 20A and the second elastomer portion 60A of the second embodiment. That is, a vaginal speculum may have a configuration in which only one of the rear portion 10 and the front portion 20 is provided with the first protrusion 112A or the second protrusion 212A.

### LIST OF REFERENCE NUMERALS

1, 1A Vaginal speculum

10, 10A Rear portion (first portion)

11, 11A First blade portion

12 First handle portion

13 Frame body portion

20, 20A Front portion (second portion)

21, 21A Second blade portion

22 Second handle portion

30 Locking member

31 Projection-bearing member

50, 50A First elastomer portion

60, 60A Second elastomer portion

111, 111A First outer face

112A First protrusion

113A First side peripheral portion

211, 211A Second outer face

212A Second protrusion

213A Second side peripheral portion

1-8. (canceled)

9. A vaginal speculum comprising:
a first portion that includes an annular frame body portion that extends in a front-rear direction, a first blade portion that extends forward from the frame body portion and is to be inserted into a vaginal cavity, and a first handle portion that extends from the frame body portion in a radial direction;
a second portion that includes a second handle portion that is opposed to the first handle portion, and a second blade portion that extends forward from the second handle portion, is opposed to the first blade portion, and is to be inserted into the vaginal cavity together with the first blade portion;
a first elastomer portion that is made of an elastomer and is arranged on a first outer face of the first blade portion, the first outer face being on a side opposite to the second blade portion; and
a second elastomer portion that is made of an elastomer and is arranged on a second outer face of the second blade portion, the second outer face being on a side opposite to the first blade portion;
wherein the second handle portion is configured to be pivotable relative to the first handle portion,
the second blade portion is configured to be pivoted relative to the first blade portion while being relatively moved away therefrom and brought close thereto, in response to pivot of the second handle portion relative to the first handle portion, and
the first elastomer portion and the second elastomer portion are arranged to prevent the first outer face of the first blade portion and the second outer face of the second blade portion from coming into contact with an inner wall of the vaginal cavity when the first blade portion and the second blade portion are inserted into the vaginal cavity.

10. The vaginal speculum according to claim 9,
wherein the first blade portion includes a first protrusion constituted by a bulging portion formed on the first outer face,
the second blade portion includes a second protrusion constituted by a bulging portion formed on the second outer face, and
the first elastomer portion is arranged on a region on the first outer face other than the first protrusion and is brought into intimate contact with a side peripheral portion of the first protrusion, and the second elastomer portion is arranged on a region on the second outer face other than the second protrusion and is brought into intimate contact with a side peripheral portion of the second protrusion.

11. The vaginal speculum according to claim 9,
wherein the first elastomer portion includes a protruding portion that protrudes forward with respect to a front end portion of the first blade portion.

12. The vaginal speculum according to claim 10,
wherein the first elastomer portion includes a protruding portion that protrudes forward with respect to a front end portion of the first blade portion.

13. The vaginal speculum according to claim 9,
wherein the second elastomer portion includes a protruding portion that protrudes forward with respect to a front end portion of the second blade portion.

14. The vaginal speculum according to claim 10,
wherein the second elastomer portion includes a protruding portion that protrudes forward with respect to a front end portion of the second blade portion.

15. The vaginal speculum according to claim 11,
wherein the second elastomer portion includes a protruding portion that protrudes forward with respect to a front end portion of the second blade portion.

16. The vaginal speculum according to claim 12,
wherein the second elastomer portion includes a protruding portion that protrudes forward with respect to a front end portion of the second blade portion.

17. The vaginal speculum according to claim 9,
wherein the first portion and the second portion are made of a hard resin or a metal.

18. The vaginal speculum according to claim 10,
wherein the first portion and the second portion are made of a hard resin or a metal.

19. The vaginal speculum according to claim 9,
wherein the first elastomer portion is formed to be inseparable from the first portion, and
the second elastomer portion is formed to be inseparable from the second portion.

20. The vaginal speculum according to claim 10,
wherein the first elastomer portion is formed to be inseparable from the first portion, and
the second elastomer portion is formed to be inseparable from the second portion.

21. The vaginal speculum according to claim 11,
wherein the first elastomer portion is formed to be inseparable from the first portion, and
the second elastomer portion is formed to be inseparable from the second portion.

22. The vaginal speculum according to claim 12,
wherein the first elastomer portion is formed to be inseparable from the first portion, and
the second elastomer portion is formed to be inseparable from the second portion.

23. The vaginal speculum according to claim 9,
wherein one of the first handle portion and the second handle portion includes a projection-bearing member that includes a main body portion extending in a front-rear direction and a projection formed on the main body, and
the other of the first handle portion and the second handle portion is provided with a through hole through which the projection-bearing member passes, and a locking member that is to be engaged with the projection to lock a pivoted position of the second blade portion relative to the first blade portion.

24. The vaginal speculum according to claim 10,
wherein one of the first handle portion and the second handle portion includes a projection-bearing member that includes a main body portion extending in a front-rear direction and a projection formed on the main body, and
the other of the first handle portion and the second handle portion is provided with a through hole through which the projection-bearing member passes, and a locking member that is to be engaged with the projection to lock a pivoted position of the second blade portion relative to the first blade portion.

25. The vaginal speculum according to claim 11,
wherein one of the first handle portion and the second handle portion includes a projection-bearing member that includes a main body portion extending in a front-rear direction and a projection formed on the main body, and
the other of the first handle portion and the second handle portion is provided with a through hole through which the projection-bearing member passes, and a locking member that is to be engaged with the projection to lock a pivoted position of the second blade portion relative to the first blade portion.

26. The vaginal speculum according to claim 12,
wherein one of the first handle portion and the second handle portion includes a projection-bearing member that includes a main body portion extending in a front-rear direction and a projection formed on the main body, and
the other of the first handle portion and the second handle portion is provided with a through hole through which the projection-bearing member passes, and a locking member that is to be engaged with the projection to lock a pivoted position of the second blade portion relative to the first blade portion.

27. The vaginal speculum according to claim 23,
wherein at least one of the projection-bearing member and the locking member is made of a soft resin.

28. The vaginal speculum according to claim 24,
wherein at least one of the projection-bearing member and the locking member is made of a soft resin.
